(19) United States
(12) Patent Application Publication
(10) Pub. No.: US 2008/0255574 A1
(43) Pub. Date: Oct. 16, 2008

(54) INSTRUMENT FOR INSERTION OF PROSTHETIC COMPONENTS

(75) Inventor: Donald W. Dye, Warsaw, IN (US)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER TECHNOLOGY, INC., Warsaw, IN (US)

(21) Appl. No.: 11/735,150

(22) Filed: Apr. 13, 2007

(51) Int. Cl. A61B 17/58

(52) U.S. Cl. 606/99

(57) ABSTRACT

An instrument configured to receive and retain a prosthetic component to facilitate the implantation of the same. In one exemplary embodiment, the instrument includes an actuator and holder secured to the actuator. The holder may include an elongate body and a head portion having a plurality of resiliently deformable arms sized and shaped to receive the prosthetic component therebetween. Additionally, the holder may be placed within a guide in the actuator along which the holder may be translated. By moving the actuator, the holder may be correspondingly translated and the resiliently deformable arms compressed to restrict translation and rotation of the prosthetic component relative to the holder.

### BACKGROUND

1. Field of the Invention

The present invention relates to a surgical instrument, and, particularly, to a surgical instrument for inserting prosthetic components.

2. Description of the Related Art

Orthopedic components, such as prostheses, are commonly utilized to repair and/or replace damaged bone and tissue in the human body. For example, hip prostheses may be implanted to replace damaged or destroyed bone in the femur and/or acetabulum and to recreate the natural, anatomical articulation of the hip joint. Additionally, prostheses may be formed as modular prostheses. Modular prostheses have several individual, distinct components which are connected together to form a final, implanted prosthesis.

By utilizing modular prostheses, a surgeon is able to create incisions which are substantially smaller than incisions required for traditional prostheses. Specifically, because modular prostheses divide a prosthesis into individual components, each individual component has a size which is less than that of the entire prosthesis. Thus, smaller incisions and smaller pathways through a patient's tissue are needed to implant a modular prosthesis.

### SUMMARY

The present invention relates to a surgical instrument, and, particularly, to an instrument for inserting prosthetic components. The instrument of the present invention is configured to receive and retain a prosthetic component therein to facilitate the implantation of the same. In one exemplary embodiment, the instrument includes an actuator and a holder secured to the actuator. The holder may include an elongate body and a head portion having a plurality of resiliently deformable arms sized and shaped to receive the prosthetic component. Further, the arms may substantially match the contour of the prosthetic component to further facilitate implantation of the same. By applying a force to the actuator, the holder may be translated and the resiliently deformable arms compressed to restrict translation and rotation of the prosthetic component relative to the holder.

In another exemplary embodiment, a plurality of heads are provided, each sized and shaped to receive a different prosthetic component. Thus, when a surgeon identifies a desired prosthetic component for implantation, the corresponding head portion is selected and attached to the holder. The holder may then be attached to the actuator and the prosthetic component received and retained therein. In another exemplary embodiment, the head portion is integrally formed and/or permanently secured to the body of the holder. In this embodiment, a plurality of holders are provided each having a head portion sized and shaped to restrict translation and/or rotation of a corresponding prosthetic component. Thus, using a single actuator or a plurality of actuators having different configurations, a surgeon may select the appropriate holder and secure the same to the actuator for implantation of a corresponding prosthetic component.

Advantageously, the present instrument allows for a surgeon to secure a prosthetic component in a fixed position and insert the same through a minimally invasive incision. Further, by sizing the head portion of the holder to substantially match the contour of the prosthetic component, the size of the prosthetic component and the head portion are substantially similar. As a result, the prosthetic component may be inserted through a smaller incision than would be necessary if the surgeon was to insert the prosthetic component by hand. Moreover, the holder substantially prevents both translation and rotation of the prosthetic component relative thereto. Thus, a surgeon can orient the prosthetic component outside of the patient's body and be ensured that the component will arrive at the implantation location in a substantially identical orientation.

In one form thereof, the present invention provides an instrument for implanting a prosthetic component, the instrument including: a holder having an elongate body defining a longitudinal axis and a head portion, the head portion including at least one resiliently deformable arm dimensioned to receive and retain the prosthetic component; and an actuator receiving the holder, the actuator including a holder actuator movable between a first position in which the at least one resiliently deformable arm of said head portion is in a first, expanded condition and a second position in which the at least one resiliently deformable arm of the head portion is in a second, contracted condition; wherein in the first, expanded condition the holder receives the prosthetic component and in the second, contracted condition the holder retains and grasps the prosthetic component.

In another form thereof, the present invention provides an instrument for inserting a prosthetic component, the instrument including: a holder having a body defining a longitudinal axis and a head portion, the head portion including an arm dimensioned to receive and retain the prosthetic component; and an actuator secured to the holder, the actuator including actuation means for moving said holder from a first, expanded condition for receipt of the prosthetic component to a second, contracted condition for retaining and grasping the prosthetic component.

In yet another form thereof, the present invention provides a method of implanting a prosthetic component, including: placing a prosthetic component within a holder having at least one resiliently deformable arm which is dimensioned to retain the prosthetic component; and actuating the holder to tighten the arm against the prosthetic component and substantially prevent rotation and translation of the prosthetic component relative to the holder.

### BRIEF DESCRIPTION OF THE DRAWINGS

The above-mentioned and other features and advantages of this invention, and the manner of attaining them, will become more apparent and the invention itself will be better understood by reference to the following description of embodiments of the invention taken in conjunction with the accompanying drawings, wherein:

FIG. 1 is perspective view of an insertion instrument according to one embodiment of the present invention;

FIG. 2 is an assembly view of the actuator shown in FIG. 1;

FIG. 3 is an assembly view of the holder shown in FIG. 1;

FIG. 4 is a plan view of the holder of FIG. 3;

FIG. 5 is a side view of the holder of FIG. 3;

FIG. 6 is a perspective view of the head portion of the holder of FIG. 3;

FIG. 7 is a perspective view of a head portion according to another exemplary embodiment;

FIG. 8 is an assembly view of a holder according to another exemplary embodiment;

FIG. 9 is a plan view of the holder of FIG. 8;

FIG. 10 is a side view of the holder of FIG. 8;

FIG. 11 is a perspective view of an insertion instrument according to another exemplary embodiment;

FIG. 12 is a plan view of the insertion instrument of FIG. 11; and

FIG. 13 is a cross-sectional view of an insertion instrument taken along line 13-13 of FIG. 12.

Corresponding reference characters indicate corresponding parts throughout the several views. The exemplifications set out herein illustrate preferred embodiments of the invention and such exemplifications are not to be construed as limiting the scope of the invention in any manner.

### DETAILED DESCRIPTION

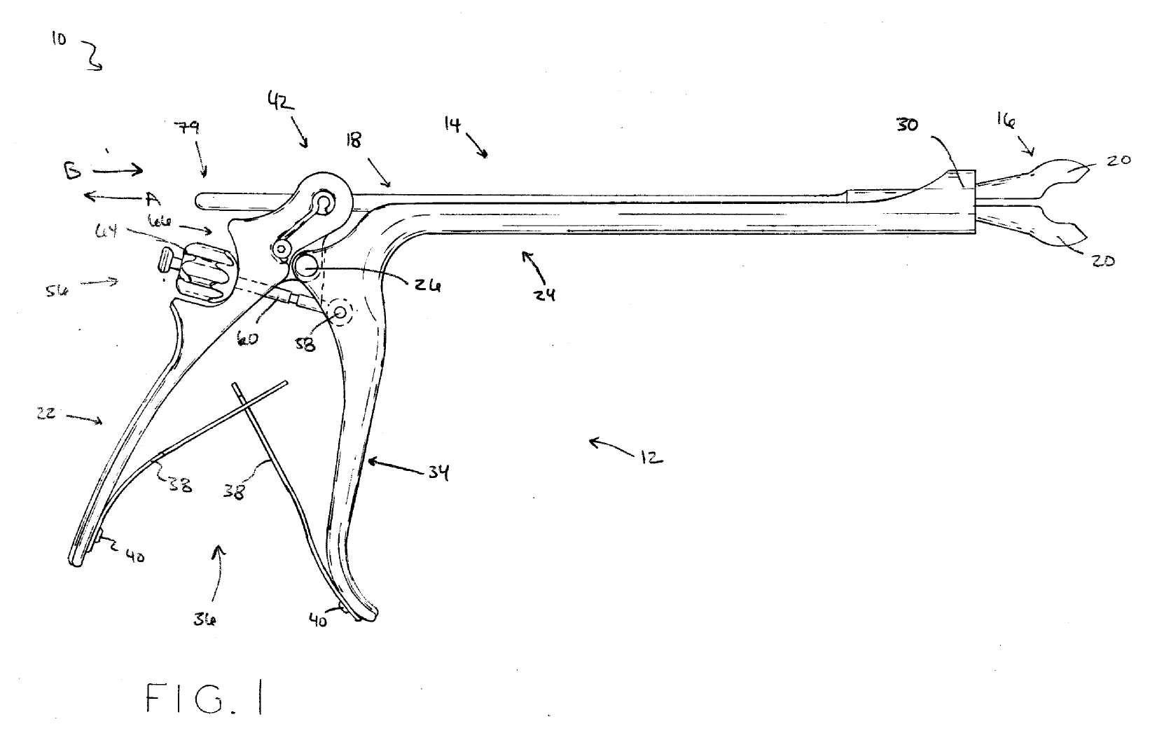

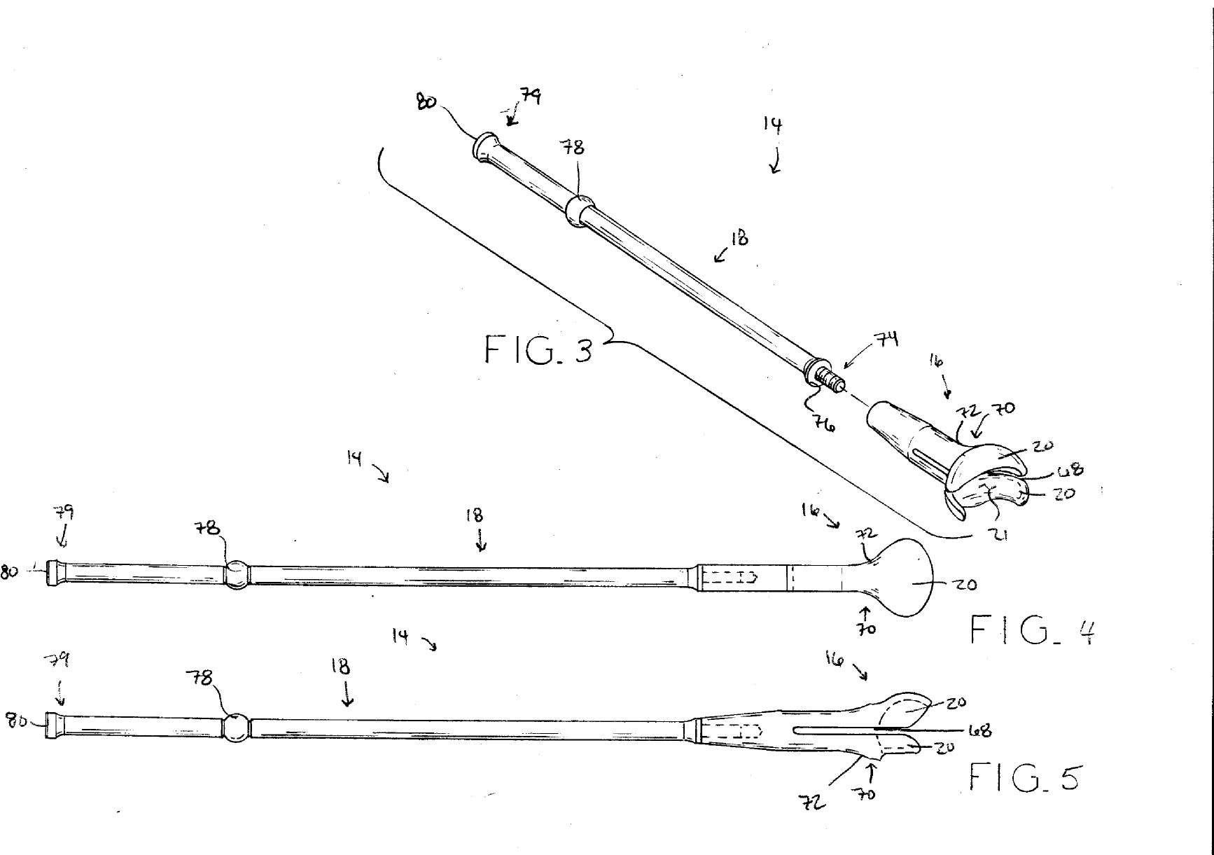

Referring to FIG. 1, instrument 10 includes actuator 12 and holder 14. As shown, holder 14 is retained by actuator 12 to substantially prevent independent translation of holder 14 relative to actuator 12. Referring to FIGS. 3-5, holder 14 includes head portion 16 and elongate body 18. Head portion 16 includes a plurality of resiliently deformable arms 20 sized and shaped to receive and retain a prosthetic component (not shown), e.g., a femoral head portion or a femoral neck, therein. By actuating a holder actuator, such as handles 22, 34 of actuator 12, holder 14 is translated in the direction of arrow A of FIG. 1, causing resiliently deformable arms 20 to tighten around a prosthetic component, as described in detail below. With a prosthetic component secured within arms 20, instrument 10 may be oriented to insert the prosthetic component into an incision in a patient and to align the same with an implantation site. Advantageously, the prosthetic component may be inserted through a smaller incision than would be necessary if the surgeon was to insert the prosthetic component by hand.

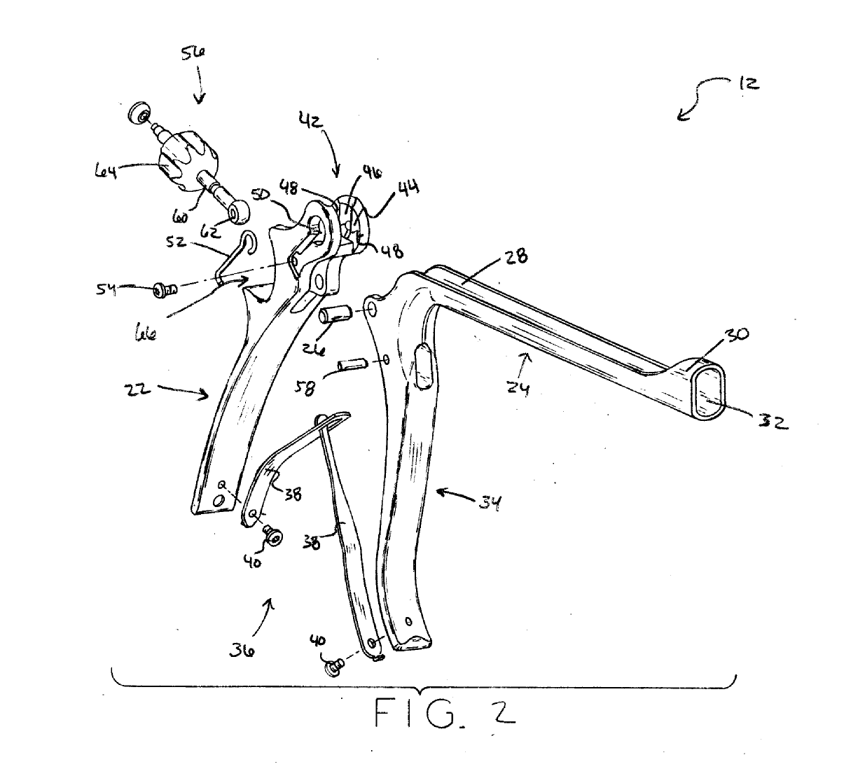

Referring to FIGS. 1 and 2, handle 22 of actuator 12 is pivotally connected to guide 24 via pivot pin 26. Guide 24 includes channel 28 (FIG. 2) extending from handle 34 to guide end 30. Aperture 32 (FIG. 2) extends through guide end 30 and is in communication with channel 28. Handle 34 extends from guide 24 in opposing relationship to handle 22. Handles 22, 34 are sized and configured to be grasped by a surgeon, such that squeezing handles 22, 34 toward one another causes the same to rotate about pivot pin 26. Biasing mechanism 36 is secured to handles 22, 34 and biases handles 22, 34 away from one another toward an unactuated position. Biasing mechanism 36 includes spring fingers 38 secured to handles 22, 34 via screws 40.

Referring to FIG. 2, handle 22 further includes holder receiving end 42 having gap 44 sized to receive holder 14 formed therein. Walls 48 define opposing sides of gap 44 and include depressions 46 formed therein. Walls 48 and depressions 46 facilitate securement of holder 14 to actuator 12 as described below. Extending through one of walls 48 and communicating with gap 44 is opening 50. Opening 50 is sized to receive retainer 52, which is secured to holder receiving end 42 of handle 22 via screw 54.

Additionally, actuator 12 includes retention mechanism 56 secured to handle 34 via pin 58. Retention mechanism 56 includes link 60 having eyelet 62 received within handle 34. Pin 58 extends through eyelet 62 and an aperture in handle 34 to secure link 60 to handle 34. Threadlingly engaged with link 60 is knob 64 which is positioned within recess 66 of handle 22. If a surgeon actuates handles 22, 34 toward one another, knob 64 may be tightened to retain handles 22, 34, and, correspondingly, actuator 12 in an actuated position. Additionally, rotation of knob 64 alone may be sufficient to cause actuation of handles 22, 34 without the need for independent actuation of handles 22, 34 by a surgeon.

Referring to FIGS. 3-5, head portion 16 of holder 14 includes resiliently deformable arms 20 separated by slot 68. Resiliently deformable arms 20 define a substantially spherically shaped seat. Specifically, interior surface 21 of each of resiliently deformable arms 20 has an at least partially spherical surface. Additionally, as described in further detail below, resiliently deformable arms 20 of head portion 16 are moveable by actuator 12 between a first, expanded condition for receipt of a substantially spherical prosthetic component therein and a second, contracted condition which retains and grasps the prosthetic component previously received. In particular, the substantially spherically shaped seat defined by arms 20 has a larger diameter in the first, expanded condition and a smaller diameter in the second, contracted condition.

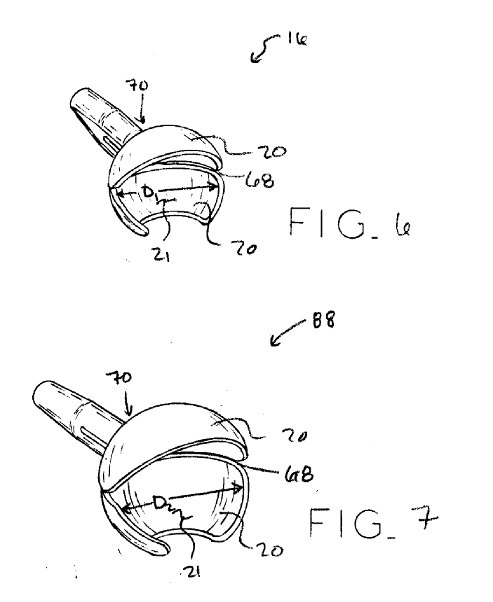

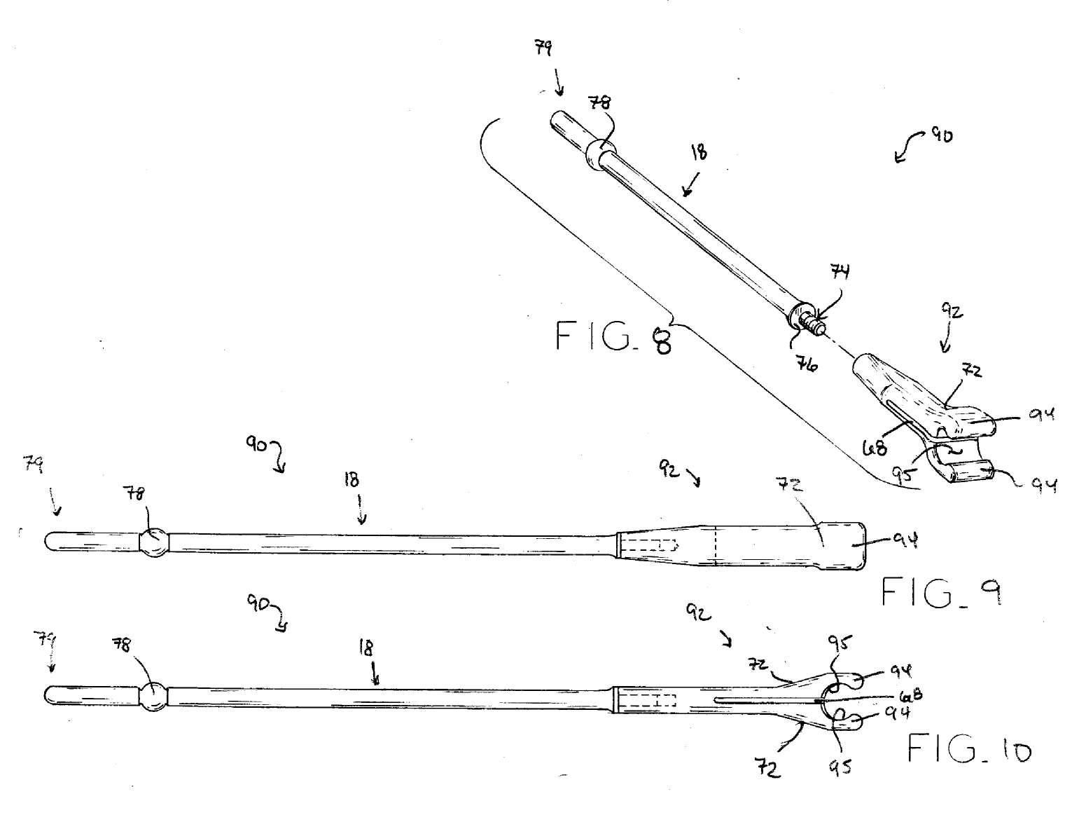

Head portion 16 may be formed of any material having a resiliency sufficient for arms 20. In one exemplary embodiment, head portion 16 is formed from polyphenylsulfone, which is commercially available from Amoco Performance Products under the tradename Radel® R. Radel® is a registered trademark of Amoco Polymers, Inc. of Alpharetta, Ga. Positioned between slot 68 and body 18 is conical section 70. Conical section 70 forms tapered surface 72 between slot 68 and elongate body 18. To secure head portion 16 to body 18, body 18 may include threaded end 74 and head portion 16 may include a corresponding internally threaded bore, shown in dashed lines in FIGS. 4 and 5. By threadingly engaging head portion 16 with threaded end 74 of body 18, head portion 16 and body 18 are secured together. Specifically, head portion 16 may be threaded on threaded end 74 of body 18 until head portion 16 contacts shoulder 76. Once in this position, the interaction of head portion 16 with shoulder 76 substantially prevents contamination from reaching threaded end 74 and the correspondingly internally threaded bore, easing sterilization of the same.

In another exemplary embodiment, head portion 16 is secured to elongate body 18 by epoxy. In this embodiment, the threads of threaded end 74 may be eliminated and the internal bore of head portion 16 filled with epoxy. By securing the components together with epoxy, the ease of sterilization of holder 14 is further advanced. In another exemplary embodiment, head portion 16 and elongate body 18 are assembly using a thermal process. For example, elongate body 18 may be heated and then inserted within an aperture formed in head portion 16. As a result of the increased temperature of elongate body 18, the area of head portion 16 defining the aperture may melt and interdigitate with an interface feature, such as knurling, formed on elongate body 18. Then, once elongate body 18 and head portion 16 cool, elongate body 18 and head portion 16 are substantially premanently secured together. In another exemplary embodiment, head portion 16 and elongate body 18 are integrally formed.

Elongate body 18 of holder 14 further includes boss 78 extending therefrom. As shown in FIGS. 3-5, boss 78 forms a substantially arcuate projection sized for receipt within gap 44 of actuator 12, as described in detail below. In another exemplary embodiment, holder 14 includes impaction surface 80, shown in FIGS. 3-5, formed at end 79 of elongate body 18 to allow a surgeon to apply an impaction force to impaction surface 80. This impaction force may facilitate seating of a prosthetic component at the desired implantation site.

As shown in FIGS. 6 and 7, instrument 10 may be configured for use with a plurality of different heads. In one exemplary embodiment, the plurality of heads, such as heads 16, 88, may be formed in different sizes corresponding to different sizes of prosthetic components. As shown in FIGS. 6-7, head portion 88 has a diameter D₂ that is larger than diameter D₁ of head portion 16. Specifically, interior surface 21 of head portion 16 defines a diameter D₁ at a predetermined distance from slot 68. Similarly, interior surfaces 21 of head portion 88 has a diameter D₂, taken at the same predetermined distance from slot 68 as diameter D₁, which is greater than diameter D₁. As described and depicted herein head portion 88 includes several features which are identical or substantially identical to corresponding features of head portion 16 and identical reference numerals have been used to identify the corresponding features therebetween. In this manner, a surgeon may select the desired prosthetic component for a particular patient and then select the appropriate head portion 16, 88, which corresponds in size to the selected prosthetic component. In another exemplary embodiment, a plurality of holders 14 are formed, as described in detail above, each of the plurality of holders 14 including one of a plurality of heads 16 of various size corresponding to the various sizes of available prosthetic components.

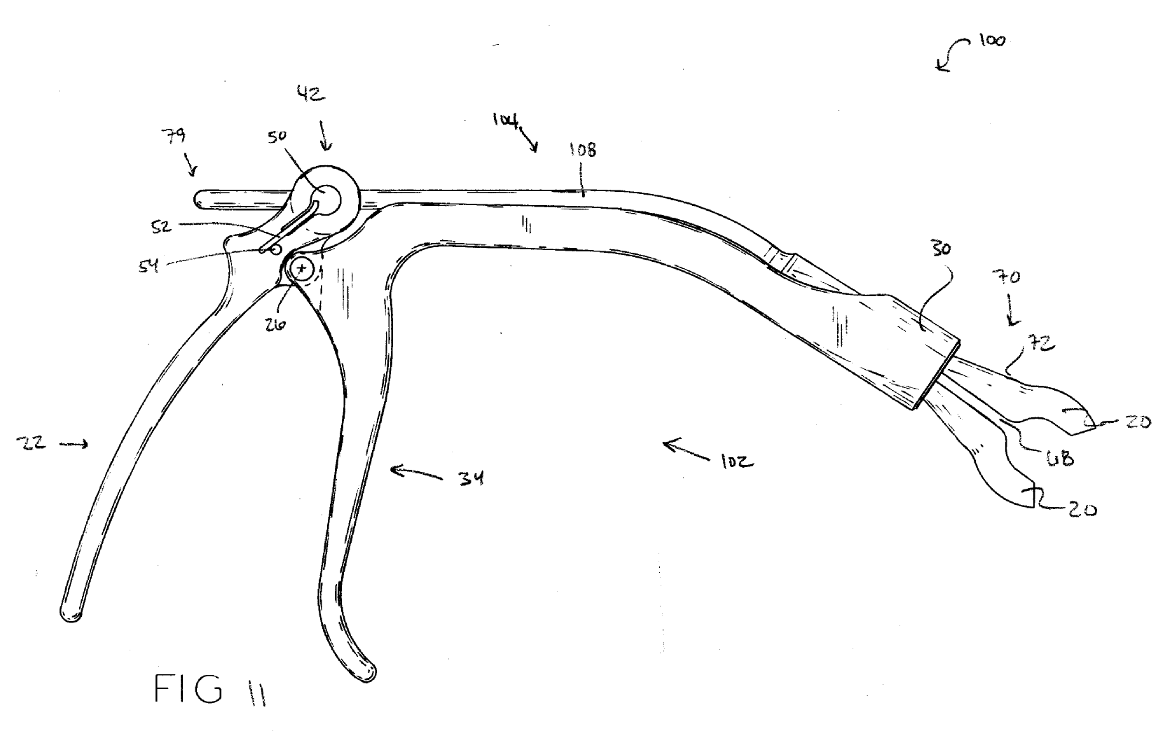

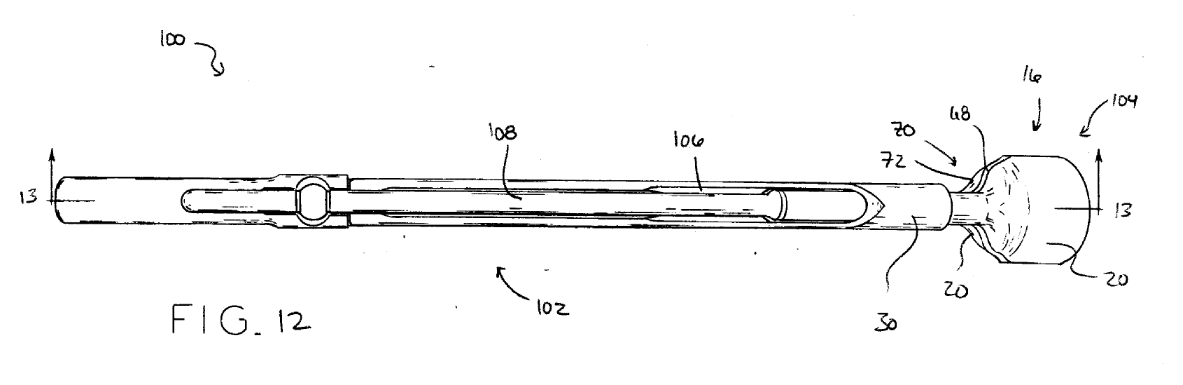

In another exemplary embodiment, shown in FIGS. 8-10, actuator 12 may be utilized in conjunction with holder 90 having head portion 92 sized and configured to substantially prevent rotation and translation of a different prosthetic component. Holder 90 of FIGS. 8-10 have several features which are identical or substantially identical to corresponding features of holder 14 and identical reference numbers have been used to identify identical or substantially identical features therebetween. Holder 90 also operates in a substantially similar manner as holder 14 of FIGS. 1 and 3-5. However, as stated above, head portion 92 and resiliently deformable arms 94 of holder 90 are configured to receive a different prosthetic component, such as a femoral neck.

Specifically, semi-cylindrical interior surfaces 95 of arms 94 define a substantially cylindrically shaped seat. Additionally, as described in further detail below with respect to holder 14, resiliently deformable arms 94 of head portion 92 are moveable by actuator 12 between a first, expanded condition for receipt of a substantially cylindrical prosthetic component therein and a second, contracted condition which retains and grasps the prosthetic component previously received. In particular, the substantially cylindrically shaped seat defined by arms 94 has a larger diameter in the first, expanded condition and a smaller diameter in the second, contracted condition.

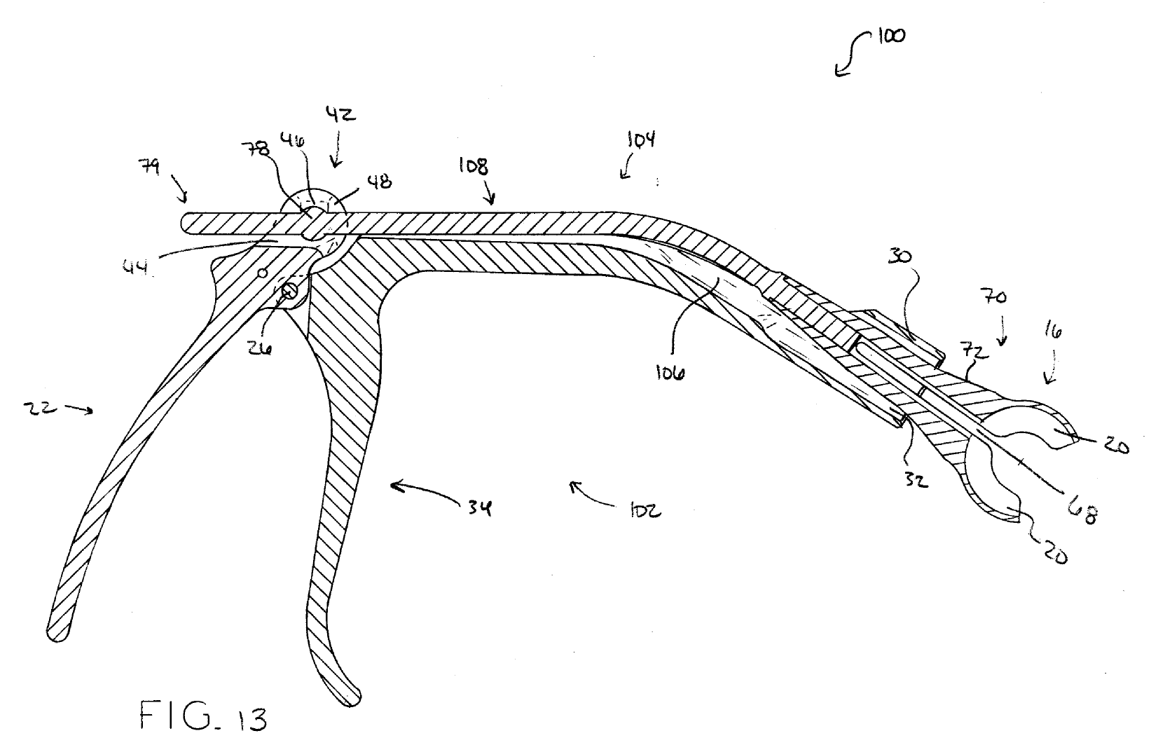

Referring to FIGS. 11-13, another exemplary embodiment of instrument 10 is depicted as instrument 100. Instrument 100 has several features which are identical or substantially identical to corresponding features of instrument 10 and identical reference numbers have been used to identify identical or substantially identical features therebetween. Instrument 100 includes actuator 102 and holder 104. Specifically, actuator 102 and holder 104 are substantially identical in use and operation to actuator 12 and holder 14. However, channel 106 of actuator 102 and body 108 of holder 104 are curved. By adding a curve to channel 106 and body 108 of actuator 102 and holder 104, respectively, the angle of access provided to the implantation site and the view of surgeon are both altered. Thus, in some procedures, the use of instrument 100 may be advantageous.

While the assembly and operation of the instruments depicted and described herein is discussed in detail below with specific reference to instrument 10, it should be understood that instrument 100 is assembled and operated in a substantially similar manner as instrument 10. Referring to FIG. 1, holder 14 is secured to actuator 12 by inserting end 79 through aperture 32 in guide end 30. Holder 14 is positioned above channel 28 and advanced toward holder receiving end 42 until boss 78 of body 18 is substantially aligned with depression 46 of holder receiving end 42. Once in this position, holder 14 is moved downwardly toward handle 22 and a sufficient force is applied to resiliently deform walls 48 and seat arcuate boss 78 against depressions 46. Body 18 is then received within channel 28 and boss 78 is retained within depressions 46 by the force of walls 48 and the additional securement force of retainer 52. Specifically, the curved portion of retainer 52 extends through opening 50 and applies a retaining force against boss 78. In this manner, independent translation of holder 14 relative to actuator 12 is substantially prevented.

With holder 14 secured to actuator 12, a surgeon may grasp handles 22, 34 and squeeze the same together. Once the force exerted by the surgeon is sufficient to overcome the biasing force of biasing mechanism 36, handles 22, 34 will pivot about pivot pin 26 and move toward one another. As this occurs, holder receiving end 42 of actuator 12 will move in the direction of arrow A (FIG. 1) causing translation of holder 14 in the same direction. As holder 14 is translated, holder 14 advances along channel 28 and through aperture 32 until tapered surface 72 of conical section 70 begins contacting guide end 30. As holder 14 is further translated in the direction of arrow A, resiliently deformable arms 20 are forced toward one another, decreasing the diameter of the portion of head portion 16 defined by arms 20. In this manner, the further that holder 14 is actuated in the direction of arrow A, the greater the force exerted by guide end 30 on resiliently deformable arms 20, and, correspondingly, a prosthetic component positioned therein.

Once a surgeon has exerted a force sufficient to retain a prosthetic component within resiliently deformable arms 20, i.e., the translation and rotation of the prosthetic component is substantially prevented, the surgeon may rotate knob 64 of retention mechanism 56 to tighten the same against handle 22. In this manner, actuator 12 is secured in the desired position and, correspondingly, the prosthetic component is secured in a predetermined position and held within resiliently deformable arms 20.

Once the prosthetic component is secured and the actuator locked by retention mechanism 56, a surgeon may manipulate instrument 10 to insert the prosthetic component within an incision in a patient and aligning the same with the implantation site. Once the prosthetic component is properly aligned, the surgeon may, if one of holders 14 including impaction surface 80 is utilized, apply an impaction force to impaction surface 80. This allows a surgeon to properly seat the prosthetic component without the need to expose additional areas of the patient's body to apply the impaction force directly to the prosthetic component. Once the prosthetic component is sufficiently seated at the implantation site, the surgeon may advance knob 64 in a direction of arrow A. As knob 64 is loosened, biasing mechanism 36 forces handles 22, 34 away from one another causing holder 14 to be translated in the direction of arrow B of FIG. 1 toward guide end 30. Once holder 14 has been sufficiently translated, such that tapered surface 72 no longer contacts guide end 30, arms 20 may be resiliently deformed to pass over and release the implanted prosthetic component from instrument 10. Instrument 10 may then be removed from the implantation site.

While this invention has been described as having a preferred design, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. An instrument for implanting a prosthetic component, the instrument comprising:
a holder having an elongate body defining a longitudinal axis and a head portion, said head portion including at least one resiliently deformable arm dimensioned to receive and retain the prosthetic component; and
an actuator receiving said holder, said actuator including a holder actuator movable between a first position in which said at least one resiliently deformable arm of said head portion is in a first, expanded condition and a second position in which said at least one resiliently deformable arm of said head portion is in a second, contracted condition;
wherein in the first, expanded condition said holder receives the prosthetic component and in the second, contracted condition said holder retains and grasps the prosthetic component.

2. The instrument of claim 1, wherein said at least one resiliently deformable arm of said head portion at least partially defines a substantially spherically shaped seat.

3. The instrument of claim 1, wherein said at least one resiliently deformable arm of said head portion at least partially defines a substantially cylindrically shaped seat.

4. The instrument of claim 1, wherein said actuator further includes a guide end and said head portion of said holder further includes a tapered surface, whereby movement of said holder actuator from said first position to said second position results in engagement of said tapered surface of said head portion with said guide end of said actuator to initiate movement of said holder between the first, expanded condition and the second, contracted condition.

5. The instrument of claim 1, further comprising a plurality of holders, each of said plurality of holders having an elongate body defining a longitudinal axis and a head portion, each said head portion of said plurality of holders including at least one resiliently deformable arm having an internal surface, said internal surfaces having respectively varying dimensions.

6. The instrument of claim 1, wherein said actuator further comprises a retention mechanism, said retention mechanism retaining said holder in said second contracted condition.

7. The instrument of claim 1, wherein said elongate body of said holder further includes an impaction surface opposite said head.

8. The instrument of claim 1, wherein said head portion is removeably secured to said elongate body of said holder.

9. The instrument of claim 8, further comprising a plurality of heads, each of said plurality of heads including at least one resiliently deformable arm having an internal surface having respectively varying dimensions to receive and retain different prosthetic components.

10. An instrument for inserting a prosthetic component, the instrument comprising:
a holder having a body defining a longitudinal axis and a head portion, said head portion including an arm dimensioned to receive and retain the prosthetic component; and
an actuator secured to said holder, said actuator including actuation means for moving said holder from a first, expanded condition for receipt of the prosthetic component to a second, contracted condition for retaining and grasping the prosthetic component.

11. The instrument of claim 10, wherein said actuation means further includes a guide end and said head portion of said holder further includes a tapered surface, whereby movement of said actuation means results in engagement of said tapered surface with said guide end to initiate movement of said holder from said first, expanded condition to said second, contracted condition.

12. The instrument of claim 10, further comprising a plurality of holders, each of said plurality of holders having a body defining a longitudinal axis and a head portion, each said head portion of said plurality of holders including an arm having an internal surface defining different seating geometry dimensioned to receive and retain a different prosthetic component, each of said plurality of holders configured for selective securement to said actuator.

13. The instrument of claim 10, wherein said body of said holder further includes an impaction surface, whereby application of a force against said impaction surface facilitates implantation of the prosthetic component.

14. The instrument of claim 10, wherein said head portion is removeably secured to said elongate body of said holder.

15. The instrument of claim 14, further comprising a plurality of head portions, each of said plurality of head portions including an arm sized to receive and retain a different prosthetic component.

16. The instrument of claim 10, wherein said head portion is substantially permanently secured to said elongate body of said holder.

17. A method of implanting a prosthetic component, comprising:
placing a prosthetic component within a holder having at least one resiliently deformable arm which is dimensioned to retain the prosthetic component; and
actuating the holder to tighten the arm against the prosthetic component and substantially prevent rotation and translation of the prosthetic component relative to the holder.

18. The method of claim 16, further comprising the step of impacting a surface on the holder to facilitate implantation of the prosthetic component.

19. The method of claim 16, further comprising the step of securing the holder to an actuator.

20. The method of claim 18, further comprising the step of actuating a handle of the actuator.

21. The method of claim 16, further comprising the step of actuating the holder in a second direction to loosen the arm and facilitate removal of the prosthetic component from the holder.
